(19) United States
(12) Patent Application Publication
(10) Pub. No.: US 2002/0091400 A1
(43) Pub. Date: Jul. 11, 2002

(54) Tongue cleaning instrument

(76) Inventor: Seenauth Harold, Ft. Myers, FL (US)

Correspondence Address: Harold Seenauth, 13606 River Forest Dr., Ft. Myers, FL, 33905, US

(21) Appl. No.: 09/756,178

(22) Filed: Jan. 8, 2001

(51) Int. Cl. A61B 17/24

(52) U.S. Cl. 606/161

(57) ABSTRACT

The invention pertains to a tongue cleaning instrument consisting of an elongated rectangular blade made of a plastic material. The blade should be stiff but also flexible. The rectangular blade has at each end thereof a handle. The handles may be separably fastened to each other so that the instrument can be used with either two hands or one hand. One of the edges of the rectangular blade is pointed. The instrument is scraped over the tongue to eliminate bad breath and to enhance the tasting of food.

### BACKGROUND OF THE INVENTION

The invention pertains to a cleaning instrument for cleaning the tongue of a person. That person can be of any age starting with about 5 years old. A thorough search of the prior art has not uncovered any such instrument. Many items have been developed or are already a long time in the market place to treat the mouth hygienically to avoid bad breath, for example, such as mouth wash, tooth paste, or flossing between the teeth. Nobody has ever thought of the fact that certain layers on the tongue can contribute to the presence of bad breath.

### SUMMARY AND OBJECTS OF THE INVENTION

An object of the invention is to alleviate the above noted problems by using a simple scraper to remove any layer that may have formed on the tongue. The layers may include bacteria and other microscopic organisms. Long term experiments have shown that the use of the scraper instrument in cleaning the tongue not only eliminates bad breath but also contributes a great deal to the enjoyment of food because the taste of the food has been greatly increased. It is a well known fact that the taste buds are located within the surface of the tongue. It stands to reason that when a microscopic organism layer is located on the tongue, the taste buds are somewhat obscured. Therefore, the present invention contributes to the hygiene of the mouth and also enhances the tasting of food. This instrument, at the point of sale should be located where other mouth and teeth treatment items are located.

### BRIEF DESCRIPTION OF THE DRAWINGS

FIG. 1 shows the cleaning instrument as it is applied to a tongue in an open position.

FIG. 2 shows the cleaning instrument as it is applied to a tongue in a closed position;

FIG. 3 is a cross section through the cleaning instrument;

FIG. 4 is another cross section through the cleaning instrument;

FIG. 5 shows a way of attaching the handles together;

FIG. 6 shows another way of attaching the handles together.

### DETAILED DESCRIPTION OF THE INVENTION

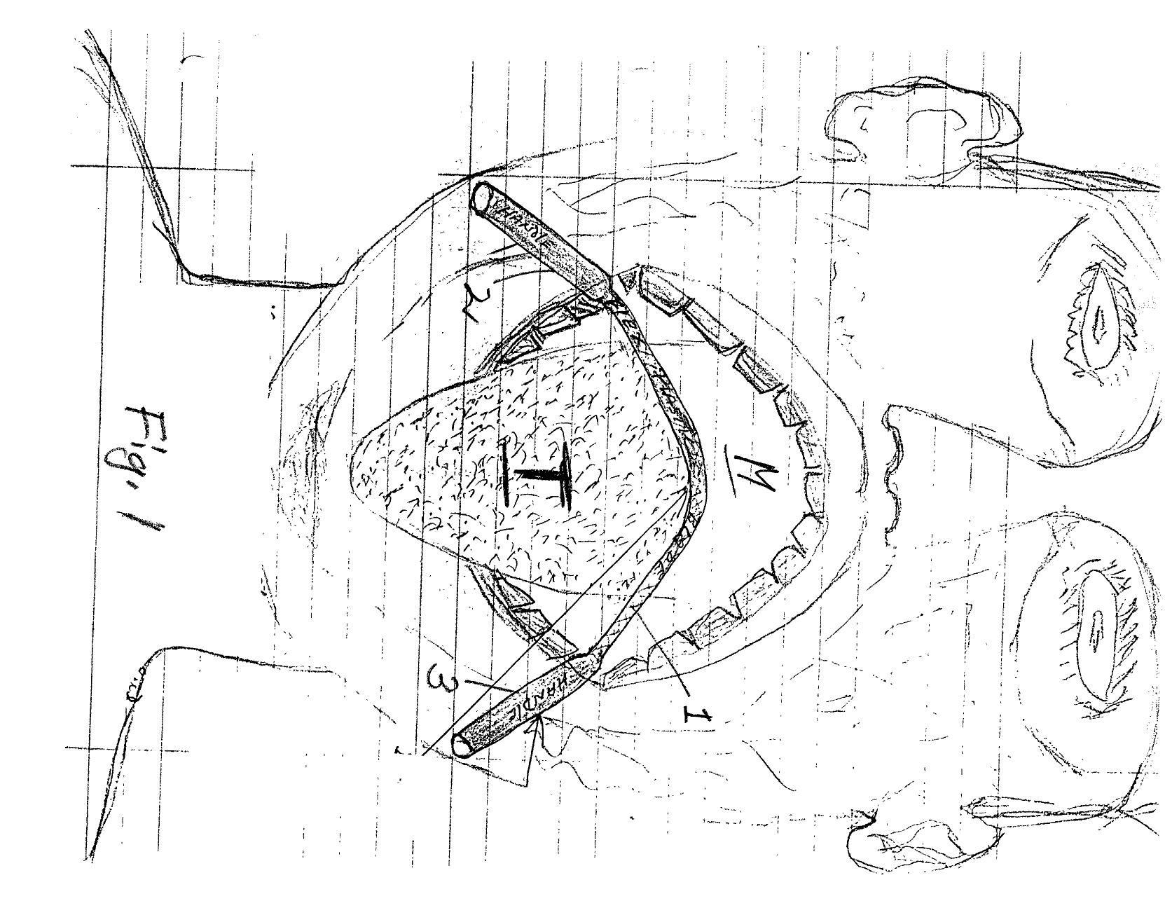

In FIG. 1 shows the cleaning instrument 1 as it is applied on a tongue T of a human's mouth M. The instrument consists of a flat and stiff but flexible elongated blade. The blade can be made of an ABC plastic material or of a polyethylene plastic material. The blade is of a rectangular cross section having various profiles which will be explained below. The blade can be easily manufactured by way of extrusion and then cut to a proper length. It is desirable that two handles 2 and 3 be applied one on each end of the blade. This will facilitate the handling of the instrument when in use. The handles can be plastic tubes that are pushed over the ends and are fastened thereto by either press fitting or by an adhesive. The cleaning instrument of FIG. 1 must be used with two hands when in use.

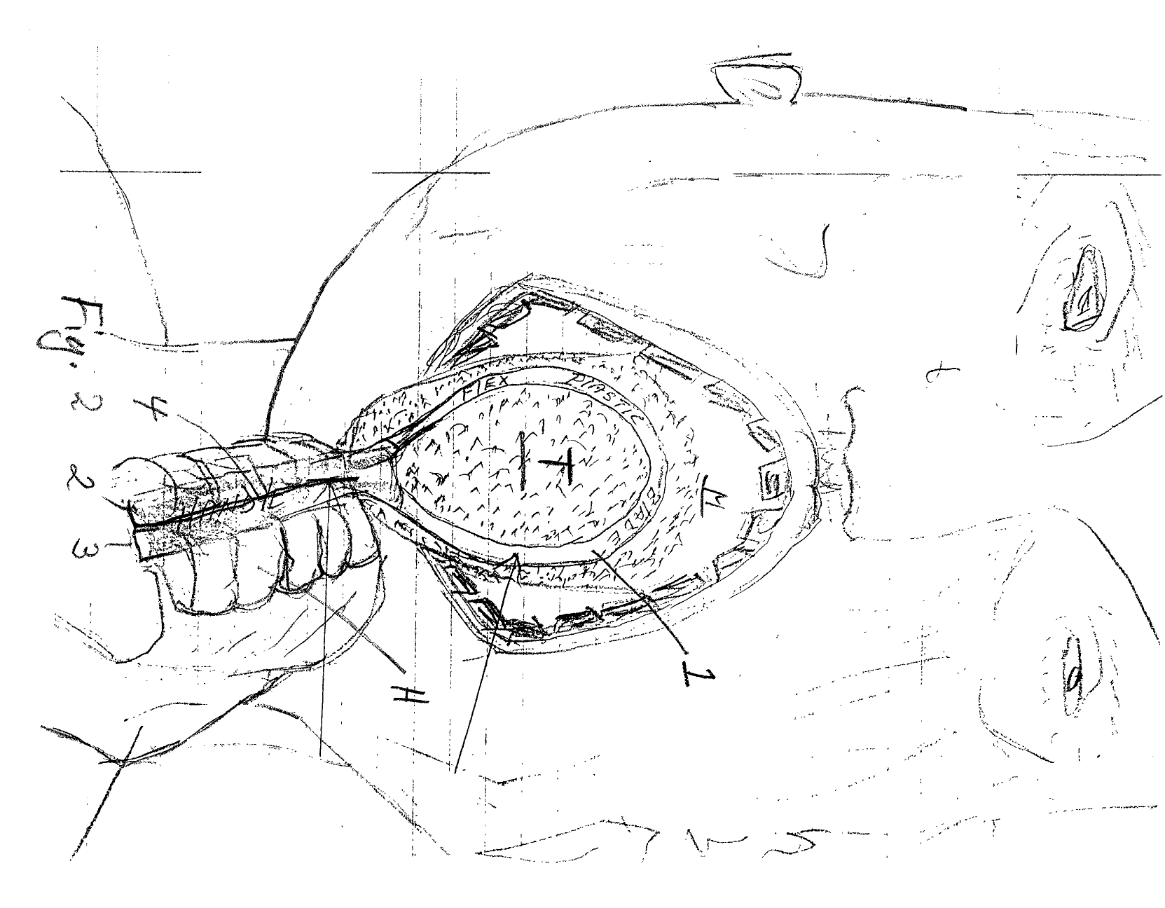

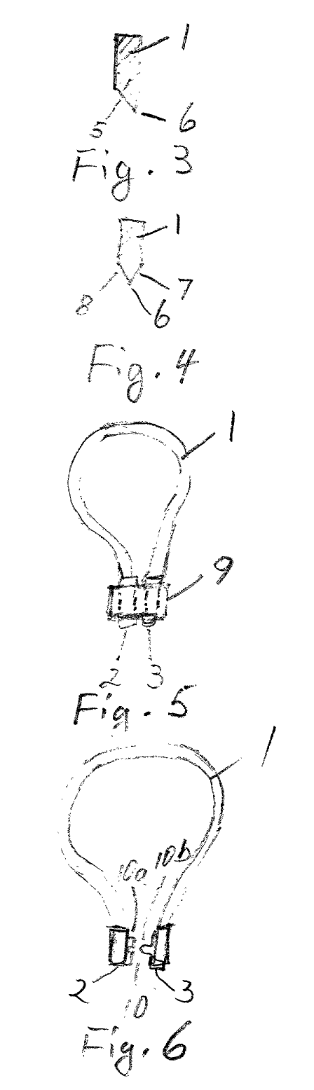

However, under certain circumstances it is desirable that the instrument can be used with one hand. FIG. 2 shows this situation. To this end, the two handles 2 and 3 are combined into one handle by separately fastening the two handles together into one handle. It is preferred that the two handles 2 and 3 can be separated from each other for either a one hand use or for a two hands use. In FIG. 2 the separable fastener consists of the hook and loop type fastening system wherein the hook strips and the loop strips can easily be fastened to each of the handles, respectively, by gluing, for example. FIG. 5 shows a simple system of separably fastening the two handles 2 and 3 by simply slipping a form fitting plastic sleeve over the two handles. Another way of fastening the two handles 2 and 3 together is by way of press buttons 10 as shown in FIG. 6. One of the handles 2, for example, receives the female part 10a and the other handle 3 receives the male part 10b. These parts can easily be pressed together and separated from each other. The parts can be attached to the respective handles by an adhesive, for example. In the description above it was mentioned that the plastic blade could be made of different profiles or cross sections. One edge of the blade could be more pointed than the other edge of the rectangular blade. This may be more desirable in that it will be effective to enhance the blade's cleaning action. FIG. 3 shows a cross section through the blade 1 wherein a slant 5 toward the other side surface of the blade 1 provides such a pointed edge. In FIG. 4 there is shown a blade 1 wherein a slants 7 and 8 from both side surfaces of the blade 1 meet in the middle of the edge to thereby form a pointed edge. The pointed edge 6 should never be sharp enough to cut the tongue which would be counterproductive to the intent of the instrument.

What I claim is:

1. A tongue cleaning instrument comprising an elongated rectangular blade made of a plastic material being stiff but still flexible, said blade having handles attached to each of the blade's ends, said handles having means thereon to be separably attached to each other.

2. The tongue cleaning instrument of claim 1, wherein said means for separably attaching is a hook and loop type fastening system.

3. The tongue cleaning instrument of claim 1, wherein said means for separably fastening is a form fitting sleeve pushed over both of said handles.

4. The tongue cleaning instrument of claim 1, wherein said means for separably fastening is a press button system.

5. The tongue cleaning instrument of claim 1, wherein said rectangular blade has one pointed edge.
